(19) United States
(12) Patent Application Publication
(10) Pub. No.: US 2025/0129435 A1
(43) Pub. Date: Apr. 24, 2025

(54) METHOD AND KIT RELATED TO LYMPHOMA, BREAST CANCER OR SUBTYPES THEREOF

(71) Applicants: DANA-FARBER CANCER INSTITUTE, INC., Boston, MA (US); The Board of Trustees of the Leland Stanford Junior University, Stanford, CA (US); Liga Nacional contra el Cáncer/Instituto de Cancerologia Guatemala, Guatemala City (GT)

(72) Inventors: Edward BRIERCHECK, Boston, MA (US); Fabiola Valvert GAMBOA, Guatemala City (GT); Oscar SILVA, Stanford, CA (US); Yaso NATKUNAM, Stanford, CA (US); Kristen STEVENSON, Boston, MA (US); David WEINSTOCK, Boston, MA (US)

(73) Assignee: Dana-Farber Cancer Institute, Inc., Boston, MA (US)

(21) Appl. No.: 18/924,535

(22) Filed: Oct. 23, 2024

Related U.S. Application Data
(60) Provisional application No. 63/592,637, filed on Oct. 24, 2023.

(51) Int. Cl. C12Q 1/6886; C12Q 1/6844

(52) U.S. Cl. CPC C12Q 1/6886; C12Q 1/6844; C12Q 2600/158; C12Q 2600/16

(57) ABSTRACT

The present disclosure provides a novel method of diagnosing lymphoma, breast cancer, a lymphoma subtype, or a breast cancer subtype in a patient, and kits for implementing the methods.

### CROSS REFERENCE TO RELATED APPLICATIONS

This application claims benefit under 35 USC 119 (e) of U.S. Application Ser. No. 63/592,637, the entire contents of which are incorporated herein by reference.

### STATEMENT REGARDING FEDERALLY SPONSORED RESEARCH

This invention was made with government support under grant no. PS00272953 awarded by the United States Department of State. The Government has certain rights in the invention.

### FIELD

The present disclosure provides a novel method of diagnosing lymphoma, breast cancer, a lymphoma subtype or a breast cancer subtype in a patient.

### BACKGROUND

Cancer is a public health crisis in low- and middle-income countries (LMICs). By 2040, approximately 80% of all cancer-related deaths will occur in these countries. Many cancers, including hematologic malignancies such as lymphoma and pediatric tumors, may be cured using low-cost chemotherapies or targeted agents.

However, especially in LMICs, most patients do not receive a diagnosis that is adequate to determine the best therapy due to the high cost of diagnostic tools and inadequate numbers of trained pathologists. Consequently, individual patients receive inadequate/incorrect therapy and health systems are unable to accurately monitor cancer incidence. For example, lymphomas of the breast are not the same as breast cancer, yet may appear indistinguishable. The ability to distinguish between breast lymphoma and breast cancer allows patients to receive appropriate treatment. Major initiatives by government agencies, non-governmental organizations (NGOs), and industry partners are seeking to increase access to therapeutics. Further, global limitations on diagnostics have limited enrollment in clinical trials to primarily high resource health systems.

Low cost and accurate diagnostic methods are thus needed globally when screening and/or diagnosing patients suspected of having cancer, lymphoma or displaying certain lymphoma specific symptoms, without the requirement for a trained pathologist or expensive confirmatory testing. Such a method would advantageously provide a primary diagnosis immediately, as opposed to requiring the use of expensive pathology for obtaining a primary diagnosis. The ability to quickly and accurately distinguish between different sub-types of lymphoma, or between lymphoma and breast cancer, using the same patient sample would represent a major advance in the art.

### SUMMARY

The present methods address at least one of these needs. Specifically, the present methods can distinguish between different sub-types of lymphoma, between different subtypes of breast cancer and/or between lymphoma and breast cancer, using a single patient sample/assay. By determining the gene expression products from the sample, and categorizing the sample into one or more diagnostic categories based upon this determination, a quick and accurate diagnoses can be provided. A quick diagnoses, in turn can allow for appropriate treatment, specific to the diagnoses, to be administered quickly. Better patient outcomes are thus provided.

In an aspect, a method of diagnosing lymphoma, breast cancer, a lymphoma subtype, or a breast cancer subtype, in a patient is provided. The method comprises: detecting the presence or absence of a one or more genes from a panel of genes associated with lymphoma, breast cancer, a lymphoma subtype, or a breast cancer subtype, in a sample from a patient; categorizing the sample into one or more diagnostic categories thereby diagnosing the lymphoma and/or lymphoma subtype, or breast cancer and/or breast cancer subtype; wherein the panel of genes comprises one or more of BACH2, BCMA, CD22, CD38, CD43, CD79a, CD138, CD200, CD274/PD-L1, CK7, CK20, Cyp141, ESR1, HER2/neu, HHV8, HBZ(HTLV), IS6110, PGR, TTF-1, LANA-1, and TDT or any combination thereof, and wherein each gene of the panel is associated with one or more diagnostic categories.

The sample may comprise a tissue volume of about 0.25 mm³ to about 5 mm³. The sample may be a formalin-fixed, paraffin-embedded (FFPE) biopsy sample. The detection may be made using multiplex PCR and standard capillary electrophoresis. The panel of genes may further comprise one or more of ALK, BCL2, BCL2A1, BCL6, BMP7, CCND1, CD244, CD44, CD5, CRBIN, DLEU1, EBER1, FCER2, FOXP1 GATA3, ICOS, ID3, IGHM, IRF4, LMO2, MAL, MKI67, MME, MS4A1, MYBL1, MYC, NCAM1, NEK6, NFKBIA, PAX5, REL, SOX8, STAT3, TBX21, TCF3, TNFRSF13B, and TNFSR8 or any combination thereof.

The diagnostic categories may comprise one or more of anaplastic large cell lymphoma, ALK− or ALK+, angioimmunoblastic T-cell lymphoma, B-lymphoblastic lymphoma, Burkitt lymphoma EBV− or EBV+, chronic lymphocytic leukemia/SLL, classical Hodgkin lymphoma, diffuse large B-cell (DLBCL), EBV+ DLBCL, extranodal NK/T cell lymphoma, grade I-IIIA follicular lymphoma, grade IIIB follicular lymphoma, HHV− or HHV+ Castleman Disease, Kaposi sarcoma, Mantle Cell lymphoma (MCL), marginal zone lymphoma (MZL), Mycosis Fungoides, nodal hyperplasia, nodular lymphocytes-predominant Hodgkin lymphoma, peripheral T-cell lymphoma NOS, plasma cell neoplasm, Plasmablastic lymphoma, T-cell lymphoblastic lymphoma, or tuberculosis.

The diagnostic categories may further comprise breast cancer subtypes ER/PR+HER2, ER/PR+HER2+, ER/PR−HER2+, or ER/PR−HER2−. In some embodiments, the diagnosing comprises generating a probability threshold, e.g., using an algorithm. In some embodiments, the probability threshold is at least 0.6. In some embodiments, if the probability threshold is met, the diagnostic accuracy of the method may be determined. In some embodiments, the diagnostic accuracy of the method is at least 94%.

The method may further comprise administering a therapeutically effective amount of treatment to the patient, wherein the treatment is selected from a therapeutic agent, radiation therapy, or surgery. In another aspect, the therapeutic agent comprises one or more of a pharmaceutical agent, a chemotherapeutic agent, an immunotherapeutic agent, or combinations thereof.

A kit is also provided and comprises: (a) one or more reagents for detecting the presence or absence of one or more genes from a panel of genes associated with lymphoma, a lymphoma subtype, breast cancer and/or breast cancer subtype, from a patient biopsy sample; instructions for measuring gene expression of the one or more genes; and (c) a classification guide for categorizing the biopsy sample into diagnostic categories; wherein the panel of genes comprises one or more of BACH2, BCMA, CD22, CD38, CD43, CD79a, CD138, CD200, CD274/PD-L1, CK7, CK20, Cyp141, ESR1, HER2/neu, HHV8, HBZ(HTLV), IS6110, PGR, TTF-1, LANA-1, and TDT or any combination thereof, and wherein each gene of the panel is associated with one or more diagnostic categories.

The panel of genes may further comprise one or more of ALK, BCL2, BCL2A1, BCL6, BMP7, CCND1, CD244, CD44, CD5, CRBIN, DLEU1, EBER1, FCER2, FOXP1 GATA3, ICOS, ID3, IGHM, IRF4, LMO2, MAL, MKI67, MME, MS4A1, MYBL1, MYC, NCAM1, NEK6, NFKBIA, PAX5, REL, SOX8, STAT3, TBX21, TCF3, TNFRSF13B, and TNFSR8 or any combination thereof.

In one aspect, the diagnostic categories comprise anaplastic large cell lymphoma, ALK− or ALK+, angioimmunoblastic T-cell lymphoma, B-lymphoblastic lymphoma, Burkitt lymphoma EBV− or EBV+, chronic lymphocytic leukemia/SLL, classical Hodgkin lymphoma, diffuse large B-cell (DLBCL), EBV+DLBCL, extranodal NK/T cell lymphoma, grade I-IIIA follicular lymphoma, grade IIIB follicular lymphoma, HHV− or HHV+Castleman Disease, Kaposi sarcoma, Mantle Cell lymphoma (MCL), marginal zone lymphoma (MZL), Mycosis Fungoides, nodal hyperplasia, nodular lymphocytes-predominant Hodgkin lymphoma, peripheral T-cell lymphoma NOS, plasma cell neoplasm, Plasmablastic lymphoma, T-cell lymphoblastic lymphoma, or tuberculosis.

In an aspect, the diagnostic categories further comprise breast cancer subtypes ER/PR⁺HER2⁻, ER/PR⁺HER2⁺, ER/PR⁻HER2⁺, or ER/PR⁻HER2⁻.

### BRIEF DESCRIPTION OF THE FIGURES

FIG. 1 is a Venn diagram showing the potential diagnoses that may be made in a large pool of patients (FIG. 1a) by a clinician suspecting a diagnosis (FIG. 1b) or actual diagnoses determined by pathology (FIG. 1c) and the potential diagnoses from biopsies (FIG. 1d).

FIG. 2 is a table listing the various machine learning approaches applied to the gene expression determinations paired with WHO standard diagnoses.

FIG. 3 is a flow chart depicts the methodology used to develop one or more diagnostic categories in an aspect.

FIG. 4 is a plot of the Spearman correlation coefficients for each gene in the panel of genes.

FIG. 5 is boxplot of the gene expression data for diagnostic samples by lymphoma subtype, wherein AGG BCL=aggressive B-cell lymphoma, DLBCL=diffuse large B-cell lymphoma FL=follicular lymphoma, HL=Hodgkin lymphoma, MCL=mantle cell lymphoma, MZL=marginal zone lymphoma, NKTCL=NK/T-cell lymphoma, TCL=T-cell lymphoma.

FIG. 6 is a boxplot of the gene expression data for relapsed samples by lymphoma subtype, wherein AGG BCL=aggressive B-cell lymphoma, DLBCL=diffuse large B-cell lymphoma FL=follicular lymphoma, HL=Hodgkin lymphoma, MCL=mantle cell lymphoma, MZL=marginal zone lymphoma, NKTCL=NK/T-cell lymphoma, TCL=T-cell lymphoma.

FIG. 7 is a heat map with hierarchical clustering using Spearman correlation in the panel of genes of Example 2, wherein AGG BCL=aggressive B-cell lymphoma, DLBCL=diffuse large B-cell lymphoma FL=follicular lymphoma, HL=Hodgkin lymphoma, MCL=mantle cell lymphoma, MZL=marginal zone lymphoma, NKTCL=NK/T-cell lymphoma, TCL=T-cell lymphoma.

FIG. 8 depicts a confusion matrix representing accuracy in classification of all cases in the validation cohort, wherein AGG BCL=aggressive B-cell lymphoma, DLBCL=diffuse large B-cell lymphoma FL=follicular lymphoma, HL=Hodgkin lymphoma, MCL=mantle cell lymphoma, NKTCL=NK/T-cell lymphoma, NM=non-malignant, TCL=T-cell lymphoma.

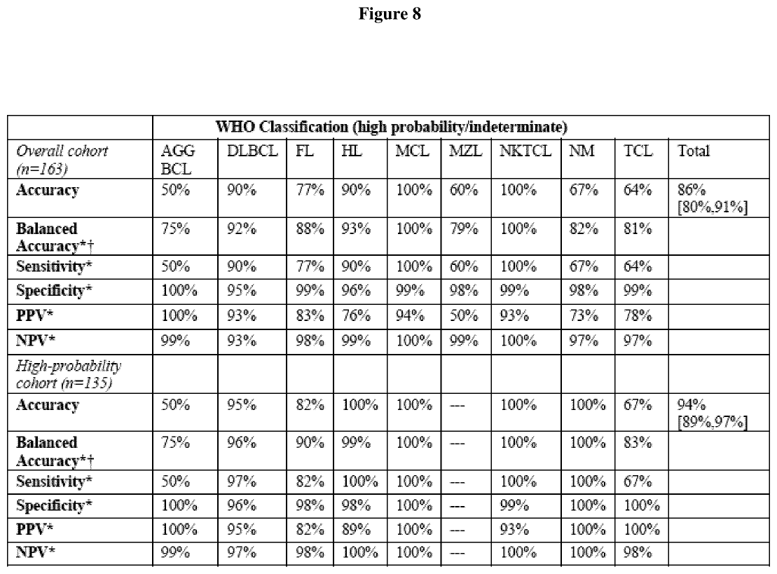

### DETAILED DESCRIPTION

Accurate diagnosis is an essential component of optimal cancer care. A novel method of diagnosing a lymphoma, breast cancer, a lymphoma subtype, or a breast cancer subtype in a patient, are provided. The methods involve detecting the presence or absence of one or more genes from a panel of genes. Each of the genes in the panel of genes is associated with one or more diagnostic categories. The samples are then categorized into the one or more diagnostic categories based on the presence or absence of the one or more genes, and thereafter the lymphoma, breast cancer, or subtype thereof is diagnosed.

Unless otherwise defined, all terms of art, notations, and other scientific terms or terminology used herein are intended to have the meanings commonly understood by those of skill in the art to which this application pertains. The following references provide one of skill with a general definition of many of the terms used in the instant disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

As used herein, the following terms have the meanings as ascribed to them below, unless specified otherwise.

Unless specifically stated or obvious from context, as used herein, the terms “a”, “an”, and “the” are understood to be singular or plural.

The term “about” is understood as within a range of normal tolerance in the art, for example within 2 standard deviations of the mean. “About” may be understood as within 10%, 9%, 8%, 7%, 6%, 5%, 4%, 3%, 2%, 1%, 0.5%, 0.1%, 0.05%, or 0.01% of the stated value. All numerical values provided herein are modified by the term about.

“Administer” refers to the direct application of an agent to a subject. A medication may be administered by ingestion, inhalation, infusion, injection, or any other means, whether self-administered or administered by a clinician or other medical professional.

The term “sample” refers to one obtained from a subject or patient. A sample includes samples of biological tissues or fluid origin, obtained, reached, or collected in vivo or in situ, that contain or are suspected of containing a polynucleotide. A biological sample may also include samples from a region of a biological subject or patient, containing precancerous or cancer cells or tissues, or diseased cells or tissues. Such samples can be, but are not limited to, blood, organs, tissues, fractions and cells isolated from mammals, including humans such as a patient, or animals. Biological samples may also include sections of biopsy samples, including tissues, for example, frozen sections taken for histological purposes. Samples may also include those that are formalin-fixed and paraffin-embedded, (FFPE). The term “diagnostic sample” refers to the first diagnosis that a patient receives for lymphoma. The term “relapse sample” refers to a patient that was previously diagnosed and received treatment, but their cancer returned, and their sample being tested is from a biopsy sample after treatment.

“Diagnostic category” refers to a set of predefined categories that are based on a combination of clinical features, such as symptoms, laboratory findings, and imaging studies. Each category represents a specific group of patients who share similar diagnostic characteristics, making it easier for clinicians to identify and diagnose the underlying condition. For example, in the field of lymphoma oncology, diagnostic categories may be used to classify patients with particular lymphoma subtypes based on their specific clinical and laboratory findings. Diagnostic categories may help clinicians to arrive at a more accurate diagnosis more quickly and efficiently, which may be especially important in cases where prompt diagnosis and treatment are critical for the patient's health and well-being. They can also facilitate the development of standardized diagnostic criteria and treatment guidelines for specific medical conditions.

“Effective amount” or “therapeutically effective amount” refers to a quantity sufficient to achieve a desired effect in a subject. For instance, this can be the amount necessary to prevent, treat, or ameliorate a disease, for example, inhibiting or suppressing cancer. In embodiments, an effective amount is the amount necessary to eliminate, reduce the size or prevent metastasis of cancer or a tumor. Efficacy is first evident in the cellular response, for which a variety of in vitro and cell assays are well-known to measure. In embodiments, an effective amount is the amount necessary to significantly inhibit or reduce cancer cell proliferation or migration, invasion or adhesion. A cellular response manifests as significantly reduced tumor size, reduced or inhibited disease progression, and/or improvement in survival in a subject. More particularly, an effective amount provides improvement in important cancer endpoints, Overall Survival (OS), Disease-Free Survival (DFS), Objective Response Rate, Complete Response Rate or Progression Free Survival (PFS). See Dept. of Health and Human Services, Food and Drug Admin, e; E. A. Eisenhauer et al., New Response Evaluation Criteria in Solid Tumours: Revised RECIST Guideline (Version 1.1), 45 Eur. J. Cancer 228 (2009). When a therapeutically effective amount is indicated, the precise amount of the treatments to be administered may be determined by a physician with consideration of individual differences in age, weight, tumor size, extent of infection or metastasis, and condition of the patient (subject).

The terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. As used herein “treatment” or “treating,” includes any beneficial or desirable effect on the symptoms or pathology of a disease or pathological condition, and may include even minimal reductions in one or more measurable markers of the disease or condition being treated. Treatment may involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “therapeutic agent” refers to any pharmaceutical agent, chemotherapeutic agent, or immunotherapeutic agent. As used herein “pharmaceutical agent” refers to any small molecule chemical compound or composition thereof. For example, one class of pharmaceutical agents is anti-inflammatory agents or drugs. Exemplary anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Other pharmaceutical agents may further include analgesics or disease-modifying anti-rheumatic drugs (DMARDs).

In some embodiments, NSAIDs may be chosen from the group consisting of ibuprofen, naproxen, naproxen sodium, Cox-2 inhibitors such as VIOXX® (rofecoxib) and CELEBREX® (celecoxib), and sialylates. Exemplary analgesics are chosen from the group consisting of acetaminophen, oxycodone, tramadol or proporxyphene hydrochloride. Exemplary glucocorticoids are chosen from the group consisting of cortisone, dexamethasone, hydrocortisone, methylprednisolone, prednisolone, or prednisone. Exemplary disease-modifying anti-rheumatic drugs (DMARDs) include azathioprine, cyclophosphamide, cyclosporine, methotrexate, penicillamine, leflunomide, sulfasalazine, hydroxychloroquine, Gold (oral (auranofin) and intramuscular) and minocycline.

Examples of other pharmaceutical agents include, but are not limited to, Thalomid® [thalidomide], Pomalyst™ [pomalidomide], or Revlimid® [lenalidomide]), a proteasome inhibitor (e.g., Velcade® [bortezomib], Ninlaro™ [ixazomib] or, a histone deacetylase (e.g., Farydak™ [panobinostat]), or a nuclear export inhibitor (e.g., Xpovio® [selinexor]). “Immunotherapy” refers to the prevention, amelioration of, or treatment of disease with substances that may stimulate an immune response. Examples of immunotherapeutic agents include but are not limited to, monoclonal antibodies or immune checkpoint inhibitors, non-specific immunotherapies, oncolytic virus therapy, T-cell therapy and cancer vaccines. In some aspects, the immunotherapeutic agent may be an antibody, including but not limited to, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, ensituximab, elotuzumab, ertumaxomab, etaracizumab, farietuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, namatumab, naptumomab, necitumumab, nimotuzumab, nofetumomab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, or zalutumumab. Other immunotherapeutic agents include, but are not limited to Empliciti™ [elotuzumab], a monoclonal antibody against CD38 (e.g., Sarclisa™ [isatuximab]), an antibody against SLAMF7 (e.g., Empliciti™ [elotuzumab]), an antibody-drug conjugate (e.g., Blenrep™ [belantamab mafodotin-blmf]), a CAR-T cell therapy such as CAR-T cells for BCMA (e.g., Abecma® [idecabtagene vicleucel]) or GPRC5D, multispecific antibodies (e.g., targeting BCMA). Any known antibody for the treatment of cancer, lymphoma, or breast cancer is contemplated herein.

“Chemotherapeutic agent” refers to drugs used to treat or prevent the progression of cancer. Illustrative examples of chemotherapeutic agents include but are not limited to alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RPS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above. Anti-hormonal agents that act to regulate or inhibit hormone action on tumors such as anti-estrogens including for example tamoxifen, raloxifene, aromatase inhibiting 4 (5)-imidazoles, 4-hydroxytamoxifen, trioxifene, keoxifene, LY117018, onapristone, and toremifene (Fareston) can also be considered chemotherapeutic agents, as can anti-androgens such as flutamide, nilutamide, bicalutamide, leuprolide, and goserelin. Other chemotherapeutic agents include, but are not limited to, Kyprolis™ [carfilzomib]), etoposide, liposomal doxorubicin, melphalan, melphalan flufenamide, bendamustine). Pharmaceutically acceptable salts, acids or derivatives of any of the above chemotherapeutic agents are also contemplated.

Any known, pharmaceutical, chemotherapeutic or immunotherapeutic agent and not listed above is contemplated. Furthermore, therapeutic agents may further be combined with radiation and surgical interventions, as deemed appropriate by a physician.

As used herein “reference” refers to a standard or control condition. In certain embodiments, a reference is a healthy subject or a subject having lymphoma. In other embodiments, a “reference” is a numeric value based or a statistical calculation, for example, a numeric value based on the average value of an analyte present in a population of normal patients or patients having lymphoma. In some embodiments, a reference is a non-malignant diagnosis.

Ranges provided herein are understood to be shorthand for all of the values within the range. For example, a range of 1 to 50 is understood to include any number, combination of numbers, or sub-range, including all decimals between numbers, from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein “subject” and “patient” are used interchangeably herein, and is meant to indicate an animal. The animal can be a mammal. The mammal may be a human or non-human mammal, such as a bovine, equine, canine, ovine, rodent, or feline.

A novel method of diagnosing lymphoma, breast cancer, a breast cancer subtype, or a lymphoma subtype in a patient is provided herein. The method allows distinction between lymphoma subtypes, breast cancer subtypes, or between lymphoma and breast cancer, for rapid, accurate diagnosis and treatment.

More specifically, the method comprises determining the presence of one or more genes from a panel of genes associated with lymphoma, breast cancer, lymphoma subtypes, and/or breast cancer subtypes in a patient sample. The sample may be a biopsy sample and may be obtained from a patient using routine collection techniques known to those of ordinary skill in the art. The sample comprises a tissue volume of about 0.25 mm³ to about 5 mm³, about 0.3 mm³ to about 4 mm³, about 0.4 mm³ to about 3 mm³, about 0.5 mm³ to about 2 mm³. In an aspect, the tissue volume of the sample is about 0.5 mm³ to about 4 mm³.

The sample may be preserved by any known preservation means for use with the methods described herein. Further, the sample may be fixed, either by physical fixation or chemical fixation, including routine fixation processes known in the art. Chemical fixation includes, but is not limited to the use of cross-linking agents, chemical dehydration or coagulation. Specific examples of chemical fixation include formalin fixation, or glutaraldehyde fixation. In an aspect, the sample is chemically fixed, e.g., the sample may be formalin-fixed and paraffin-embedded (FFPE). Physical fixation includes freezing or boiling, including for example, cryofixation, and in one aspect the biopsy sample is physically fixed.

The step of detecting the presence of one or more genes from a panel of genes involves processing of samples to extract nucleic acids, which may be subsequently analyzed using techniques such as a chemical ligation probe-based assay (CLPA) for detecting gene expression, multiplex PCR and standard capillary electrophoresis, in order to identify gene signatures and quantify the levels of nucleic acid expression for specific genes within the sample. In an aspect, DNA, RNA, or mRNA may be extracted from the sample, and measured to determine the presence or absence of one or more genes of a gene panel associated with lymphoma and/or breast cancer, and that may differentiate clinically relevant lymphoma and/or breast cancer subtypes.

The panel of genes may include any combination of one or more genes described herein. In one aspect, the panel of genes comprises BACH2, BCMA, CD22, CD38, CD43, CD79a, CD138, CD200, CD274/PD-L1, CK7, CK20, Cyp141, ESR, HER2/neu, HHV8, HBZ(HTLV), IS6110, PGR, TTF-1, LANA-1, TDT and any combination thereof. In another aspect, the panel of genes further comprises one or more of ALK, BCL2, BCL2A1, BCL6, BMP7, CCND1, CD244, CD44, CD5, CRBIN, DLEU1, EBER1, FCER2, FOXP1 GATA3, ICOS, ID3, IGHM, IRF4, LMO2, MAL, MKI67, MME, MS4A1, MYBL1, MYC, NCAM1, NEK6, NFKBIA, PAX5, REL, SOX8, STAT3, TBX21, TCF3, TNFRSF13B, or TNFSR8. Any known genes associated with lymphoma, breast cancer and/or subtypes of either are also contemplated for inclusion in the panel of genes. In some aspects, the panel of genes may be leveraged to distinguish between breast cancer subtypes. After a diagnosis of breast cancer, the expression of three breast cancer genes ESR1 (estrogen receptor), PGR (progesterone receptor) and ERBB2 (HER2/Neu receptor) have direct implications on patient treatment. Current immunohistochemistry and fluorescent in situ hybridization (“FISH”) methods are prohibitively expensive in LMICs. mRNA expression of ESR1 has shown to be more predictive of response to anti-estrogen therapy than immunohistochemistry. Thus, the addition of breast cancer associated genes renders the panel of genes useful as a primary diagnostic not only for suspected lymphoma, but also for breast cancer.

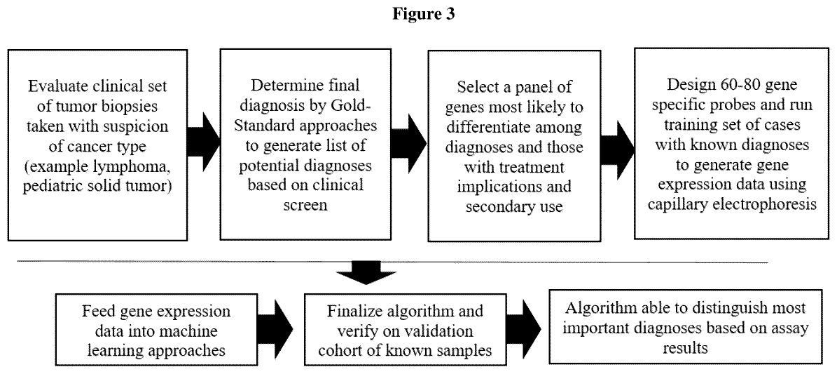

Various combinations of any number of genes from the gene panel may be used to create diagnostic categories. By using available treatment guidance, the epidemiology of lymphoma subtypes and by further matching gene expression data to known diagnoses, diagnostic categories may be defined and used to identify the subtype of lymphoma or breast cancer based on the genes of the gene panel detected (or not detected) in the sample. Machine learning and statistical modeling may be used to assist in the establishment of the diagnostic categories such that accurate classification of the sample and/or distinction between lymphoma and breast cancer is provided. Accordingly, in one aspect, the present method allows for the diagnosis of lymphoma and/or breast cancer, and their subtypes, by classification of samples according to diagnostic categories. FIG. 3 depicts the overall methodology used to develop the panel of genes for the method described herein.

In an aspect, the diagnostic categories comprise lymphoma or lymphoma subtypes, and/or breast cancer or breast cancer subtypes. In such aspects, the diagnostic categories comprise one or more of anaplastic large cell lymphoma, ALK− or ALK+, angioimmunoblastic T-cell lymphoma, B-lymphoblastic lymphoma, Burkitt lymphoma EBV− or EBV+, chronic lymphocytic leukemia/SLL, classical Hodgkin lymphoma, diffuse large B-cell (DLBCL), EBV+DLBCL, extranodal NK/T cell lymphoma, grade I-IIIA follicular lymphoma, grade IIIB follicular lymphoma, HHV− or HHV+Castleman Disease, Kaposi sarcoma, Mantle Cell lymphoma (MCL), marginal zone lymphoma (MZL), Mycosis Fungoides, nodal hyperplasia, nodular lymphocytes-predominant Hodgkin lymphoma, peripheral T-cell lymphoma NOS, plasma cell neoplasm, Plasmablastic lymphoma, T-cell lymphoblastic lymphoma, or tuberculosis.

Once samples have been analyzed for the presence or absence of one or more genes from the gene panel, the collected data may be input into an algorithm and the sample classified into diagnostic categories.

In an embodiment, a probability threshold is set. If the probability threshold is reached for a sample, a diagnosis of lymphoma, breast cancer, a lymphoma subtype, or a breast cancer subtype may be generated for the patient. If the probability threshold is not reached for a sample, a diagnosis is not generated and the patient may be referred for standard pathology testing. The use of artificial intelligence and algorithms to perform any data analysis, characterization or classification, statistical analysis or predictive modeling, is contemplated herein. In some embodiments, the probability threshold is about 0.3 to about 0.9, 0.4 to about 0.8. 0.5 to about 0.7, or about 0.6.

Confidence in the diagnoses may be ascertained by calculating a diagnostic accuracy as follows: accuracy=(true positive+true negative)/(true positive+true negative+false positive+false negative). In one aspect, the diagnostic accuracy comprises at least about 90%, 91%, 92%, 93%, 94%, 95%, 96%, 97%, 98%, 99%, or about 100%. In another aspect, the diagnostic accuracy comprises about 90% to about 93%, about 92% to about 94%, about 93% to about 95%, about 94% to about 96%, about 97% to about 99%, or about 98% to about 100%. In another aspect, the diagnostic accuracy comprises at least about 94%.

In some embodiments, the probability threshold is from 0.3 to 0.9 and the diagnostic accuracy is at least about 90%. In some embodiments, the probability threshold is from 0.4 to 0.8 and the diagnostic accuracy is at least about 91%. In some embodiments, the probability threshold is from 0.5 to 0.7 and the diagnostic accuracy is at least about 92%. In some embodiments the probability threshold is at least about 0.6 and the diagnostic accuracy is at least 94%.

Once a diagnosis is made, physicians may use the information to perform further confirmatory diagnostics, if needed. Advantageously, physicians may also begin to prescribe a therapy to a patient, and begin treating the identified lymphoma, breast cancer and/or lymphoma or breast cancer subtype, more quickly than if conventional pathological testing was performed. Thus, in an aspect, the method comprises treating the lymphoma, breast cancer, breast cancer subtype or lymphoma subtype. In such aspects, the method further comprises administering to the patient a therapeutically effective amount of one or more of a therapeutic agent, or prescribing radiation therapy, surgery or combination thereof. In one aspect, the therapeutic agent may comprise one or more of a pharmaceutical agent, chemotherapeutic agent, or immunotherapeutic agent, or combinations thereof, alone or in further combination with prescribed radiation therapy and/or surgery.

Many types of lymphoma are either curable or highly responsive to various therapeutic treatments, including but not limited to, CVP (cyclophosphamide, vincristine, and prednisone) or CHOP (cyclophosphamide, doxorubicin, vincristine, and prednisone) chemotherapy for follicular, marginal zone, mantle cell, peripheral T-cell, and DLBCL,CODOX m (cyclophosphamide, vincristine, doxorubicin, and methotrexate) or hyper-CVAD (hyperfractionated cyclophosphamide, vincristine, doxorubicin, and dexamethasone) chemotherapy for Burkitt or B-lymphoblastic lymphoma, ABVD (doxorubicin, bleomycin, vinblastine, and dacarbazine) chemotherapy for HL, and SMILE (dexamethasone, methotrexate, ifosfamide, L-asparaginase, and etoposide) chemotherapy for extranodal NKTCL. Any known treatments for lymphoma and various lymphoma subtypes are contemplated herein.

Breast cancers include a variety of types, including hormone-receptor-positive breast cancer, HER2-positive breast cancer, or triple negative breast cancer. Hormone-receptor positive cancers may include both estrogen and/or progesterone receptor negative or positive subtypes. Therapies directed to the breast cancer subtypes are available and are contemplated herein, including but not limited to estrogen therapy, including ovarian function suppression, estrogen receptor modulators (SERMs) or aromatase inhibitors, chemotherapy, immunotherapy including but not limited to trastuzumab (anti-HER2), surgical interventions, or combinations thereof. Any known breast cancer therapy, including therapies for ER/PR and/or HER2 subtypes breast cancer are contemplated herein.

Kits are also provided, and comprise one or more reagents for detecting one or more genes from a panel of genes associated with lymphoma and/or breast cancer, from a patient sample. Kits may include containers for staining samples and the transcriptional assays described herein. In some aspects, kits may further include instructions for use in accordance with the methods of this disclosure and instructions for generating diagnostic categories. The kit may further comprise a description of treatments suggested for a patient as suitable for treatment based on identifying whether the subject has a particular lymphoma subtype or breast cancer. In one aspect, is a kit comprising, (a) one or more reagents one or more reagents for detecting the presence or absence of one or more genes from a panel of genes associated with lymphoma, a lymphoma subtype, breast cancer and/or breast cancer subtype, from a patient sample; (b) instructions for detecting gene expression of the one or more genes; and (c) a classification guide for categorizing the sample into diagnostic categories; wherein the panel of genes comprises one or more of BACH2, BCMA, CD22, CD38, CD43, CD79a, CD138, CD200, CD274/PD-L1, CK7, CK20, Cyp141, ESR1, HER2/neu, HHV8, HBZ(HTLV), IS6110, PGR, TTF-1, LANA-1, and TDT or any combination thereof.

### EXAMPLES

### Example 1

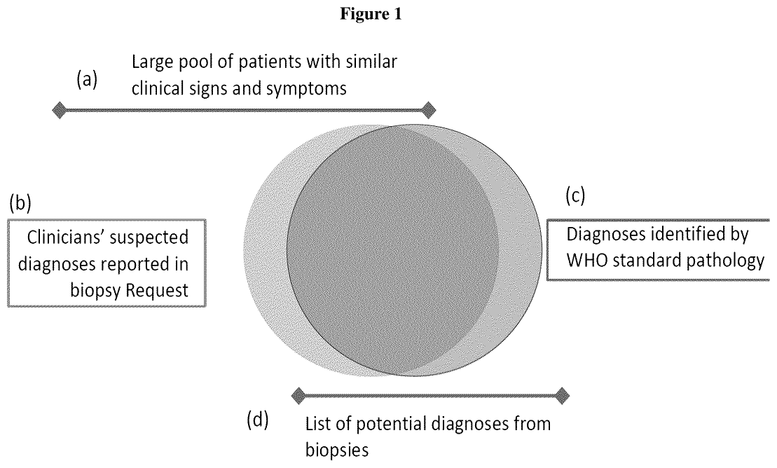

Physician reported suspected diagnoses (also called a differential diagnosis) that present with broadly similar signs and symptoms (FIG. 1a) from patients were selected and reviewed based on past biopsy requests (FIG. 1b). 670 samples were evaluated to determine, by World Health Organization pathology standards, what the true diagnosis was (FIG. 1c). This included one of the clinician's initial diagnostic suspicions or other diseases that present with similar symptoms. A list was generated with a finalized list of potential diagnoses (FIG. 1d).

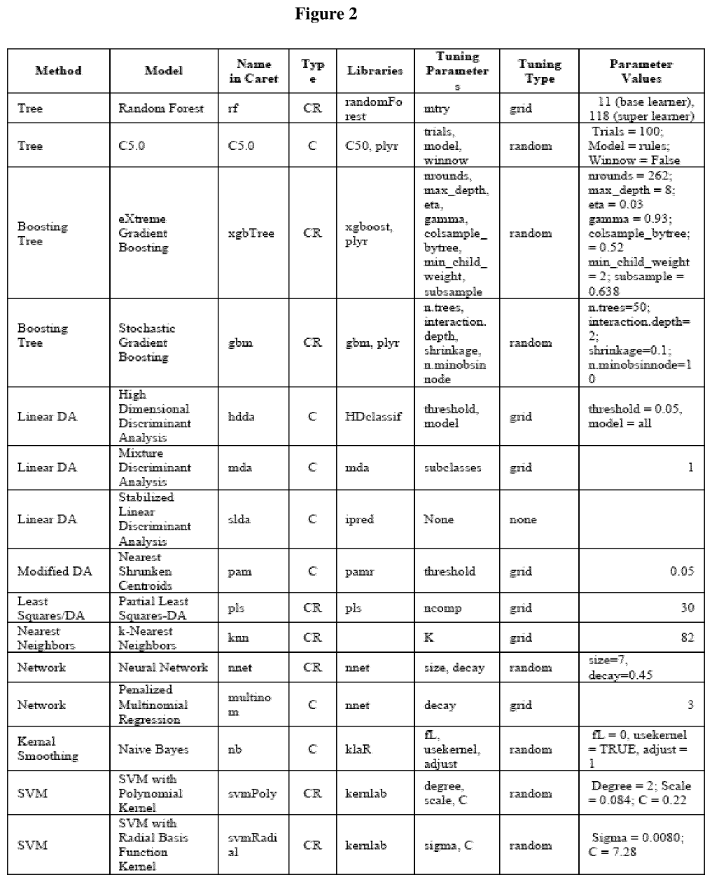

These potential diagnoses were then matched to gene expression data generated by a panel of genes most likely to differentiate the different potential diagnosis. More specifically, a gene expression assay was used to assess a panel of genes that could differentiate clinically relevant lymphoma subtypes, or distinguish between lymphoma and breast cancer. Diagnostic groups were prioritized that may affect treatment and prognosis. During the training phase of the assay design process, formalin-fixed, paraffin-embedded tissue sections were obtained from several hundred patient biopsies. Biopsies were subjected to multiplex PCR and standard capillary electrophoresis to determine the presence or absence of one or more genes from the pre-designed gene panel. These determinations were paired with the WHO standard diagnoses of the entire training cohort and were input with multiple machine learning approaches (FIG. 2) to generate a diagnostic algorithm.

This diagnostic approach was validated on an independent cohort of samples. The end product was a diagnostic algorithm that was able to distinguish the most important diagnostic groups based on determination of the presence or absence of one or more of the genes from the panel of genes.

TABLE 1
Treatment strategies based on diagnosis among cases of suspected lymphoma

| Diagnostic | | | Prognostic |
|---|---|---|---|
| Category | First-line therapy | Second-line therapy | Value |
| Diffuse large | Stage I/II: RCHOP + ISRT(Bulky | Transplant Candidate: DHAP, | |
| B-Cell | disease) | GDP, ICE (all +/− rituximab) | |
| lymphoma, NOS | Stage III/IV: RCHOP | Not transplant candidate: GemOx +/− | |
| | | rituximab, polatuzumab | |
| | | vedotin-piiq ± bendamustine ± | |
| | | rituximab, tafasitamab-cxix + | |
| | | lenalidomide | |
| EBV + diffuse | Stage I/II: RCHOP + ISRT(Bulky | Transplant Candidate: DHAP, | Poor |
| large | disease) | GDP, ICE (all +/− rituximab) | prognostic |
| B-cell | Stage III/IV: RCHOP | Not transplant candidate: GemOx +/− | group |
| lymphoma | | rituximab, polatuzumab | |
| | | vedotin-piiq ± bendamustine ± | |
| | | rituximab, tafasitamab-cxix + | |
| | | lenalidomide | |
| High grade B-cell | RCHOP, DA-EPOCH-R, | Transplant Candidate: DHAP, | Poor |
| lymphoma | RHyperCVAD, | GDP, ICE (all +/− rituximab) | prognostic |
| | R-CODX-MR-IVAC | Not transplant candidate: GemOx +/− | group |
| | | rituximab, polatuzumab | |
| | | vedotin-piiq ± bendamustine ± | |
| | | rituximab, tafasitamab-cxix + | |
| | | lenalidomide | |
| Nodular | Early stage: ABVD +/− ISRT | Rituximab + bendamustine, RDHAP | |
| lymphocyte- | Intermediate stage: ABVD, | | |
| predominant | BV-AVD | | |
| Hodgkin lymphoma | Advanced stage: BEACOPP, | | |
| | RCHOP | | |
| Classical Hodgkin | Stage I/II: ABVD +/− ISRT | Brentuximab vedotin + | |
| lymphoma | Stage III/IV: ABVD, BV-AVD, | bendamustine | |
| | BEACOPP | | |
| Plasma cell | Subtype specific | | |
| neoplasm | | | |
| Plasmablastic | daEPOCH +/− bortezomib | | |
| lymphoma | | | |
| Angioimmunoblastic | Stage I-II: ISRT + multi-agent | Belinostat, pralatrexate, | |
| T-cell | chemothrapy | romidepsin. Additional options if | |
| lymphoma | Stage III-IV: | transplant eligible DHAP, DHAX, | |
| | −CD30+: BV-CHP | ESHAP, GDP, GemOx, ICE | |
| | −CD30− CHOEP, CHOP, da- | | |
| | EPOCH | | |
| Peripheral T-cell | Stage I-II: ISRT + multi-agent | Belinostat, pralatrexate, | GATA3 |
| lymphoma, NOS | chemothrapy | romidepsin. Additional options if | expressing |
| | Stage III-IV: | transplant eligible DHAP, DHAX, | worse |
| | −CD30+: BV-CHP | ESHAP, GDP, GemOx, ICE | prognosis |
| | −CD30− CHOEP, CHOP, da- | | |
| | EPOCH | | |
| T-cell lymphoblastic | Philadelphia positive | Nelarabine ± etoposide + | |
| lymphoma | AYA: EsPhALL | cyclophosphamide | |
| | Adult: TKI + hyper-CVAD | | |
| | Philadelphia negative | | |
| | AYA: CALGB 10403, COG | | |
| | AALL0232, COG AALL0434, | | |
| | DFCI | | |
| | ALL | | |
| | Adult: CALGB 8811 Larson | | |
| | regimen, | | |
| | GRAALL-2005, Hyper-CVAD | | |
| Anaplastic large cell | BV-CHP | | Improved |
| lymphoma, | | | prognosis |
| ALK+ | | | over ALKA |
| ALKAnaplastic | BV-CHP | | |
| large cell | | | |
| lymphoma, | | | |
| ALK− | | | |
| Extranodal NK/T- | Localized: RT + DeVIC | Pembrolizumab, Nivolumab | |
| cell lymphoma | Advanced: modified SMILE, | | |
| | PGEMOX, | | |
| | DDGP, AspaMetDex | | |
| Other T-cell | Subtype specific | | |
| lymphoma | | | |
| (primary cutaneous, | | | |
| mycosis | | | |
| fungoides, CD30+ | | | |
| lymphoproliferative | | | |
| disorders of | | | |
| the skin) | | | |
| Grade I-IIIA | Stage I/II: ISRT +/− anti-CD20, | Bendamustine + Obinutuzumab, | |
| follicular lymphoma | observation | CHOP + anti-CD20, CVP + anti- | |
| | Stage III/IV: Bendamustine + | CD20, Lenalidomide + rituximab | |
| | anti-CD20, | | |
| | CHOP, CVP, | | |
| | lenalidomide + rituximab | | |
| Grade IIIB | Stage I/II: RCHOP + ISRT(Bulky | Transplant Candidate: DHAP, | |
| follicular | disease) | GDP, ICE (all +/− rituximab) | |
| lymphoma | Stage III/IV: RCHOP | Not transplant candidate: GemOx +/− | |
| | | rituximab, polatuzumab | |
| | | vedotin-piiq ± bendamustine ± | |
| | | rituximab, tafasitamab-cxix + | |
| | | lenalidomide | |
| Chronic | SLL Stage I: RT | Alternative first line | |
| lymphocytic | SLL Stage II-IV or CLL with | | |
| leukemia/small | treatment | | |
| lymphocytic | indication: Acalabrutinib ± | | |
| lymphoma | Obinutuzumab, Ibrutinib, | | |
| | Venetoclax + | | |
| | Obinutuzumab, Zanubrutinib | | |
| B-cell | Philadelphia positive | Blinatumomab | |
| lymphoblastic | AYA: EsPhALL | | |
| lymphoma | Adult: TKI + hyper-CVAD | | |
| | Philadelphia negative | | |
| | AYA: CALGB 10403, COG | | |
| | AALL0232, DFCI ALL | | |
| | Adult: CALGB 8811 Larson | | |
| | regimen, | | |
| | GRAALL-2005, Hyper-CVAD | | |
| Burkitt | Low risk: CODOX-M + | daEPOCH-R, RICE, RIVAC | |
| lymphoma | rituximab, | | |
| | daEPOCH, HyperCVAD | | |
| | High risk: CODOX-M alternating | | |
| | with | | |
| | IVAC, HyperCVAD, | | |
| | daEPOCH-R | | |
| Mantle cell | Stage I/II: ISRT, Bendamustine + | Acalabrutinib, Ibrutinib ± | |
| lymphoma | rituximab, VR-CAP, RCHOP, | rituximab, Zanubrutinib, | |
| Stage | Lenalidomide + rituximab, | Lenalidomide + rituximab (if BTK | |
| | (all +/− ISRT) | inhibitor is contraindicated) | |
| | Stage II bulky, III, IVL: | | |
| | Transplant: RDHAP, | | |
| | RCHOP/RDAP, | | |
| | NORDIC, HyperCVAD, | | |
| | Rituximab, bendamustine | | |
| | followed by rituximab, | | |
| | high-dose cytarabine | | |
| | Not transplant candidate: | | |
| | Bendamustine + rituximab, VR- | | |
| | CAP, | | |
| | RCHOP, lenalidomide + rituximab | | |
| Marginal zone | Nodal: | Bendamustine + Obinutuzumab, | |
| lymphoma | Stage I/II: ISRT +/− anti-CD20, | ibrutinib, zaubrutinib, | |
| | observation | lenalidomide + rituximab | |
| | Stage III/IV: Bendamustine + | | |
| | rituximab, | | |
| | CHOP, CVP | | |
| | Other site specific treatment for | | |
| | localized | | |
| | disease | | |
| Castleman's | Unicentric: surgery (if resectable), | Unicentric: surgery, ISRT, | |
| disease | RT, rituximab | rituximab | |
| | (+/−prednisone, +/−cyclophosphamide), | (+/−prednisone, +/−cyclophosphamide), | |
| | embolization | embolization, | |
| | Mulitcentric: | −HIV−/HHV8−: | |
| | −HIV−/HHV8−: siltuximab | Siltuximab/tocilizumab | |
| | −HIV+/−/ | Multicentric: alternative primary | |
| | HHV8+: | regimen | |
| | rituximab ± liposomal | | |
| | doxorubicinq ± | | |
| | prednisone | | |
| | Multicentric + organ failure: | | |
| | RCHOP, | | |
| | RCVAD, RCVP, R-liposomal | | |
| | doxorubicin | | |
| Tuberculosis | Pending drug resistance | | |
| Kaposi Sarcoma | ART if HIV + liposomal | | |
| | doxorubicin | | |
| Benign hyperplasia | Observation | | |

Samples were obtained from patients at Instituto Nacional de Cancerolgía (INCAN) hospital in Guatemala presenting with symptoms consistent with lymphoma. Demographics of the patients are presented in Table 2a. Tissue samples were divided in half, with one half of each sample being sent to DxTerity Genomics for measuring the expression of 37 genes, and the other half of each sample being sent to Stanford for cancer classification according to the World Health Organization standard (Table 2b).

TABLE 2a

| | Agg | | | | | | | Non- | |
|---|---|---|---|---|---|---|---|---|---|
| | BCL | DLBCL | FL | HL | MCL | MZL | NKTCL | malignant | TCL |
| N | 10 | 262 | 50 | 97 | 63 | 23 | 54 | 45 | 39 |
| Median age | 38 | 63 | 59 | 38 | 62 | 53 | 41 | 48 | 56 |
| (range) | (18-60) | (15-98) | (30-85) | (8-90) | (39-89) | (22-78) | (15-84) | (19-79) | (15-87) |

Sex (%)

| | | | | | | | | | |
|---|---|---|---|---|---|---|---|---|---|
| Female | 5 (50) | 140 (53) | 26 (52) | 43 (44) | 14 (22) | 15 (65) | 18 (34) | 18 (40) | 11 (28) |
| Male | 5 (50) | 119 (45) | 21 (42) | 53 (55) | 49(78) | 8(35) | 32(59) | 7(16) | 27(69) |
| Unknown | 0(0) | 3(1) | 3(6) | 1(1) | 0(0) | 0(0) | 4(7) | 20(44) | 1(3) |

Stage (%)

| | | | | | | | | | |
|---|---|---|---|---|---|---|---|---|---|
| I | 0(0) | 51 (19) | 6(12) | 19(20) | 1(2) | 5(22) | 18(33) | 6(13) | 8(21) |
| II | 0(0) | 60 (23) | 11(22) | 31(32) | 10(16) | 4(17) | 20(37) | 5(11) | 7(18) |
| III | 2(20) | 57(22) | 14(28) | 23(24) | 19(30) | 10(43) | 0(0) | 4(9) | 5(13) |
| IV | 5(50) | 41(16) | 6(12) | 9(9) | 25(40) | 2(9) | 5(9) | 0(0) | 9(23) |
| Unknown | 3(30) | 53(20) | 13(26) | 15(15) | 8(13) | 2(9) | 11(20) | 30(37) | 9(23) |

B symptoms (%)

| | | | | | | | | | |
|---|---|---|---|---|---|---|---|---|---|
| Yes | 4(40) | 88(34) | 11(22) | 49(51) | 30(48) | 8(35) | 15(28) | 1(2) | 11(28) |
| No | 3(30) | 112(43) | 15(30) | 47(48) | 30(48) | 3(13) | 16(30) | 2(4) | 13(33) |
| Unknown | 4(40) | 94(36) | 22(44) | 33(34) | 23(37) | 18(78) | 18(33) | 12(27) | 16(41) |

Bulky (%)

| | | | | | | | | | |
|---|---|---|---|---|---|---|---|---|---|
| Yes | 3(30) | 113(43) | 15(30) | 47(48) | 30(48) | 3(13) | 16(30) | 2(4) | 13(33) |
| No | 4(40) | 94(36) | 22(44) | 33(34) | 23(37) | 18(78) | 18(33) | 12(27) | 16(41) |
| Unknown | 3(30) | 55(21) | 13(26) | 17(18) | 10(16) | 2(9) | 20(37) | 31(69) | 10(26) |

Patient characteristics: Aggresive B-cell lymphoma = Agg BCL, Diffuse large B cell lymphoma = DLBCL (including germinal center (GCB), non-germinal center (non-GCB), or not otherwise specified (NOS), Follicular Lymphoma = FL, Hodgkin Lymphoma = HL, Mantle Cell Lymphoma = MCL, Marginal Zone Lymphoma = MZL, Natural Killer/T-cell lymphoma = NKTCL, T-cell lymphoma = TCL, or non-malignant.

TABLE 2b
WHO standard pathology diagnosis (first column)
and diagnostic category (second column) with
total cases of each diagnosis column)

| WHO Classification | CLPA Diagnostic Category | N |
|---|---|---|
| B lymphoblastic | Agg BCL | 5 |
| leukemia/lymphoma | | |
| Burkitt lymphoma | Agg BCL | 5 |
| Diffuse large B cell lymphoma, | DLBCL | 68 |
| germinal center type | | |
| Diffuse large B cell lymphoma, | DLBCL | 152 |
| non-germinal center type | | |
| Diffuse large B cell lymphoma, | DLBCL | 7 |
| NOS | | |
| EBV positive diffuse large B | DLBCL | 5 |
| cell lymphoma, NOS | | |
| High grade B cell lymphoma, | DLBCL | 15 |
| NOS | | |
| High grade B cell lymphoma, | DLBCL | 2 |
| Myc failed | | |
| High grade B cell lymphoma | DLBCL | 9 |
| with Myc and BCL2 or BCL6 | | |
| T cell histocyte rich large B cell | DLBCL | 5 |
| lymphoma | | |
| Follicular lymphoma, grade 1-2 | FL | 40 |
| Follicular lymphoma, grade 3 | FL | 11 |
| Classic Hodgkin lymphoma | HL | 91 |
| Nodular lymphocyte- | HL | 6 |
| predominant Hodgkin | | |
| lymphoma | | |
| Mantle cell lymphoma | MCL | 63 |
| Marginal zone lymphoma | MZL | 23 |
| Extranodal NK/T cell | NKTCL | 54 |
| lymphoma, nasal type | | |
| Anaplastic large cell lymphoma, | TCL | 7 |
| ALK-negative | | |
| Anaplastic large cell lymphoma, | TCL | 6 |
| ALK-positive | | |
| Angioimmunoblastic T cell | TCL | 1 |
| lymphoma | | |
| CD30 positive | TCL | 2 |
| lymphoproliferative disorder | | |
| Mycosis Fungoides | TCL | 1 |
| Nodal peripheral T cell | TCL | 2 |
| lymphoma with T follicular | | |
| helper phenotype | | |
| Peripheral T cell lymphoma, | TCL | 19 |
| NOS | | |
| Primary cutaneous CD4+ | TCL | 1 |
| small/medium T-cell | | |
| lymphoproliferative disorder | | |

Nonmalignant cases

| | | |
|---|---|---|
| Acute inflammation with | Nonmalignant | 1 |
| ulceration | | |
| Angiomyomatous hamartoma | Nonmalignant | 1 |
| Chronic gastritis | Nonmalignant | 1 |
| Chronic inflammation | Nonmalignant | 2 |
| Dermatitis | Nonmalignant | 1 |
| Fibroconnective tissue | Nonmalignant | 2 |
| Fragmented lymphoid tissue | Nonmalignant | 1 |
| Granulomatous inflammation | Nonmalignant | 1 |
| Non-lesional tissue identified | Nonmalignant | 1 |
| Progressive transformation of | Nonmalignant | 1 |
| germinal centers | | |
| Reactive follicular hyperplasia | Nonmalignant | 29 |
| Reactive lymphoid infiltrate | Nonmalignant | 1 |
| Reactive lymphoid hyperplasia | Nonmalignant | 1 |
| Normal skin | Nonmalignant | 1 |
| | Total Diagnosis | 645 |

A gene expression assay (DxTerity®) targeting a panel of genes and normalizer genes (which may control the total amount of nucleic acid isolated for appropriate comparisons), (ISY1,WDR55, ACTB, TBP) was performed using between 9-100 ng of extracted nucleic acids from formalin-fixed, paraffin-embedded (FFPE) biopsies obtained from patients identified in Table 2b.

TABLE 3
Lymphoma panel of genes and associated diagnostic categories

| Gene/Protein | Diagnostic Category(ies) |
|---|---|
| FOXP1 | ABC-DLBCL vs GCB-DLBCL¹⁻⁴ |
| | BL and molecular high-grade vs GCB-DLBCL |
| TNFRSF13B/ | ABC-DLBCL vs GCB-DLBCL and PMBCL |
| CD267/TACI | MYC+/BCL2+ vs MYC−/BCL2− DLBCL |
| | NMZL vs FL and reactive lymph node |
| IGHM | ABC-DLBCL vs GCB-DLBCL and PMBCL |
| IRF4/MUM1 | ABC-DLBCL and PMBCL vs GCB-DLBCL |
| | DLBCL vs Burkitts |
| | NMZL vs FL and reactive lymph node |
| BCL6 | GCB-DLBC vs ABC-DLBCL |
| | FL vs NMZL |
| | AITL vs ALCL, ATLL, NKTCL, PTC-NOS |
| | ALK+ vs ALK− ALCL |
| NEK6 | GCB-DLBCL and PMBCL vs ABC DLBCL |
| | AITL vs ALCL, ATLL, NKTCL, PTC-NOS |
| LMO2 | GCB-DLBCL and PMBCL vs ABC-DLBCL and BL |
| | FL and reactive lymph node vs NMZL |
| | Reactive lymph node, cHL and DLBCL vs FL |
| MYBL1/AMYB | GCB-DLBCL, vs ABC-DLBCL and PMBCL |
| | BL vs DLBCL |
| ALK | ALK+ expression improves prognosis vs |
| | ALK− in ALCL and is therapeutic target |
| | Poor prognosis in DLBCL |
| BCL2 | DLBCL vs BL |
| | DLBCL vs PMBCL |
| | AITL vs ALCL, ATLL, NKTCL/T, PTCL-NOS |
| BCL2A1/BFL1 | PMBCL vs HL and DLBCL |
| | DLBCL vs BL |
| | AITL vs ALCL, ATLL, NKTCL, PTCL-NOS |
| BMP7 | BL vs DLBCL |
| CCND1 | MCL vs SLL and MZL |
| CD244/NKR2B4 | NKTCL vs PTCL-NOS |
| | NKTCL vs AITL, ATLL, ALCL |
| | CT-PTCL vs ALK+ ALCL, ATLL, AITL |
| CD44 | PMBCL, DLBCL vs BL |
| | ABC-DLBCL vs GCB-DLBCL |
| | NMZL vs FL |
| DLEU1 | BL vs DLBCL |
| CD5 | SLL and MCL vs NMZL and FL |
| CRBN | Potential biomarker for imides |
| EBER | NKTCL, DLBCL, HL, AITL and potential biomarker |
| FCER2/CD23 | PMBCL vs DLBCL |
| | SLL vs MCL, MZL |
| | AITL vs ALCL, ATLL, NK/T, PTCL-NOS |
| GATA3 | PTCL-NOS with adverse prognosis |
| | HL (cell lines) vs normal B cells, BL, FL, CLL |
| | ATLL vs ALK+ ALCL, ATLL, AITL, CT-PTCL |
| ICOS | AITL vs ALCL, ATLL, NKTCL, PTCL-NOS |
| | PTCL vs NKTCL |
| ID3 | AITL vs ALCL, ATLL, NKTCL, PTC-NOS |
| | BL vs DLBCL |
| MAL | PMBCL vs DLBCL |
| MKI67 | MCL poor prognosis |
| | Aggressive versus indolent |
| MME/CD10 | GCB-DLBC vs ABC-DLBCL and BL |
| | FL vs NMZL |
| MS4A1/CD20 | Mature B-cell lymphomas |
| MYC/c-MYC | BL and poor prognosis DLBCL |
| | NKTCL vs normal peripheral blood NK cells |
| NCAM1/CD56 | NKTCL vs PTCL |
| NFKBIA | DLBCL vs BL |
| PAX5 | B-cell malignancies |
| REL/c-Rel | PMBCL versus DLBCL |
| SOX8 | AITL vs ALCL, ATLL, NKTCL, PTCL-NOS |
| STAT3 | DLBCL vs BL |
| TBX21/TBET | NKTCL vs PTCL |
| | PTCL-NOS with better prognosis |
| TCF3/E2A | BL and molecular high-grade vs DLBCL |
| TNFRSF8/CD30 | ALCL and subset of other PTCL and CTCL |
| | PMBCL vs DLBCL |
| TdT | Distinguish lymphoblastic lymphoma from Burkitt |
| CD34 | Lymphoblastic lymphoma |
| CK7 | Adenocarcinoma screen |
| CK20 | Metastatic GI carcinoma screen |
| ER | Breast cancer |
| PR | Breast cancer |
| HER2/neu | Breast cancer |
| TTF-1 | Lung adenocarcinoma |
| CD38 | Expressed plasma cell disorders, daratumumab target |
| CD138 | Plasmablastic lymphoma, plasma cell disorders |
| CD22 | CLL/SLL |
| CD43 | T-cell lymphoma, mantle cell lymphoma, SLL, |
| | Burkitt. Variable in MZL, DLBCL |
| CD200 | B-CLL |
| CD274/PDL1 | Eligibility for immunotherapy |
| BCMA | Expressed at mRNA level in MM, T-ALL, B-ALL, |
| | CLL/SLL, HL |
| BACH2 | Gene expression shows upregulation in CLL compared |
| | to CD5+ B-cell subsets though in others like |
| | DLBCL |
| CD79a | IHC decreased in CLL compared to other lymphomas, |
| | but similar transcript. Marker for B cells in |
| | anaplastic |
| LANA-1 | Kaposi other HHV8 positive tumors |
| (HHV8) | |
| IS6110 | Tuberculosis |
| Cyp141 | Tuberculosis |
| HBZ (HTLV) | Adult T-cell leukemia/lymphoma (HBZ) |
| Normalizer | ISY1, ACTB, TBP |
| genes | |

Samples were prepared with a total of 20 μm sliced sections formalin-fixed, paraffin-embedded tissue. Nucleic acids were then isolated in a single tube and extracted using the Promega ReliaPrep™ FFPE total RNA extraction kit (catalog #Z1002). The PCR Thermal Cycler Protocol began as follows: a hot start at 95° C. for 2 min; 3-step cycling; denaturation at 95° C. for 10 seconds; annealing at 61° C. for 20 seconds; extension at 72° C. for 20 seconds; followed by 30 cycles; extension at 72° C. for 5 min; holding for 4° C. until use in next step.

Multiplex PCR with chemical ligation was used to analyze the resulting samples. Each gene of interest had a probe sequence split into two parts with either the forward or reverse universal primer sequences on each probe pair, as well as a unique combination of stuffer sequence and fluorophores (FAM/NED/VIC/PET) corresponding to each gene. When two paired probes bound, they underwent a spontaneous chemical ligation reaction resulting in an oligonucleotide ligation product. The probes were designed so that the ligation product from each gene target would generate a unique pre-determined size and color combination. Because only oligonucleotide probes that are ligated and bound to two adjacent cDNA sequences were amplified, the number of oligonucleotide probe ligation products was a measure of the number of target gene products in a sample. This allowed for the rapid measurement of expression for multiple genes. Samples were run on standard capillary electrophoresis machines. Normalized expression values of each gene were calculated by subtracting the mean of the log 2 normalizer signals from the log 2 signal of the response gene. FFPE curls of the samples were independently reviewed by a hematopathologist and classified using the World Health Organization (WHO) classification of lymphoid neoplasms to provide the following diagnostic categories: anaplastic large cell lymphoma, ALK− or ALK+, angioimmunoblastic T-cell lymphoma, B-lymphoblastic lymphoma, Burkitt lymphoma EBV− or EBV+, chronic lymphocytic leukemia/SLL, classical Hodgkin lymphoma, diffuse large B-cell (DLBCL), EBV+DLBCL, extranodal NK/T cell lymphoma, grade I-IIIA follicular lymphoma, grade IIIB follicular lymphoma, HHV− or HHV+Castleman Disease, Kaposi sarcoma, Mantle Cell lymphoma (MCL), marginal zone lymphoma (MZL), Mycosis Fungoides, nodal hyperplasia, nodular lymphocytes-predominant Hodgkin lymphoma, peripheral T-cell lymphoma NOS, plasma cell neoplasm, Plasmablastic lymphoma, T-cell lymphoblastic lymphoma, or tuberculosis.

### Statistical Methods

RStudio version 1.1.463 with R version 3.5.1 was used for analysis. Fifteen candidate statistical models based on diagnostic samples were built using a 70% training and 30% validation split with 10-fold cross-validation and 5 repeats using the repeated CV option in the trainControl function within in the caret (Classification And Regression Training) package. A relapse sample data set with 38 patient samples was used as a test set.

A multiClassSummary option was selected with classProb=TRUE in the function trainControl. Balanced models were included as candidates by selecting the sampling option as downsampling, upsampling, or smote (package DMwR v0.4.1) also in the trainControl function. The data were centered and scaled using the preprocess option in the train function. A grid search was used for parameter tuning for models with one parameter, but a random search was initiated for models with 2 or more, followed by a grid search if needed. For each tunegrid, a minimum tunelength option of 50 different parameter settings was selected for each parameter for those with one tuning parameter. For those with 3 or more tuning parameters, at least 50 random parameter settings were attempted. The optimization metric used was logarithmic loss (the metric option=logLoss in the train function in R). This metric measures the classification performance where the prediction input is a probability value which quantifies the accuracy of a classifier by penalizing false classifications. Whereas accuracy is the proportion of correct predictions, log loss considers the certainty with which a model makes a correct or incorrect prediction.

A variety of candidate statistical models were selected based on the techniques described in the art (See, for example, Hastie, et al., The Elements of Statistical Learning. New York, NY, USA: Springer New York Inc., 2001 and selected from the possible statistical models available within caret (Classification And REgression Training, https://topepo.github.io/caret/available-models.html). Model building techniques including tree-based classification methods (random forest=rf, C5.0), boosting tree algorithms (extreme or stochastic gradient boosting, xgbtree, gbm), linear methods for classification using discriminant analysis (lda, hdda, slda, pls (partial least squares), mda), nearest neighbor classification (knn), neural networks (multinom, nnet), shrunken centroids (pam), and support vector machines (svmPoly, svmRadial).

A staged classification approach was used to return the final class label for each sample. In the first stage an ensemble model stacking approach was used. Fourteen of the 15 candidate models (naïve Bayes was excluded due to low and unstable resampling accuracy) were used as “base learners” and the class probabilities from each of these were then used as predictors in a random forest (“super learner”). An ensemble approach was considered to mitigate overfitting, increase call confidence, and utilize a variety of modeling approaches for optimization of the potential parameter spaces.

Tables 4a and 4b show the number of samples in each diagnostic category in the entire cohort, as well as in the training, validation, and test cohorts.

TABLE 4a
Lymphoma and Control Samples (Stage 1)

| | Diagnostic* | Training* | Validation* | Relapse |
|---|---|---|---|---|
| | N | 70% | 30% | Test |
| Total | 560 | 397 | 163 | 39 |
| Aggressive BCL | 9 | 7 | 2 | 1 |
| DLBCL | 232 | 163 | 69 | 19 |
| FL | 44 | 31 | 13 | 6 |
| HL | 71 | 50 | 21 | 6 |
| MCL | 56 | 40 | 16 | 5 |
| MZL | 18 | 13 | 5 | 1 |
| NK-TCL | 49 | 35 | 14 | 1 |
| TCL | 39 | 28 | 11 | 0 |
| Non-malignant | 42 | 30 | 12 | 0 |

*Diagnostic = total number of cases identified within each diagnostic category.
*Training = number of cases (70% of diagnostic) that were used for training the algorithm.
*Validation = total number of case (30% of diagnostic) that were used to validate the accuracy of the trained algorithm.

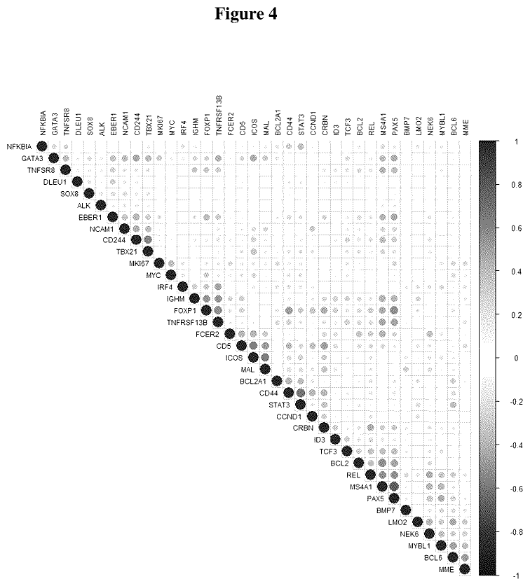

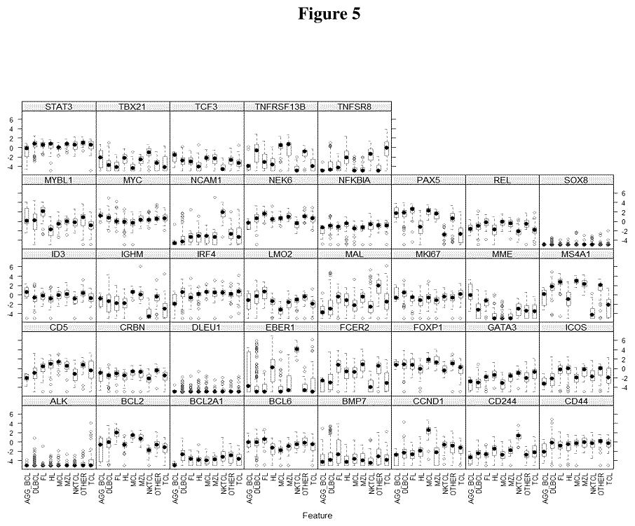

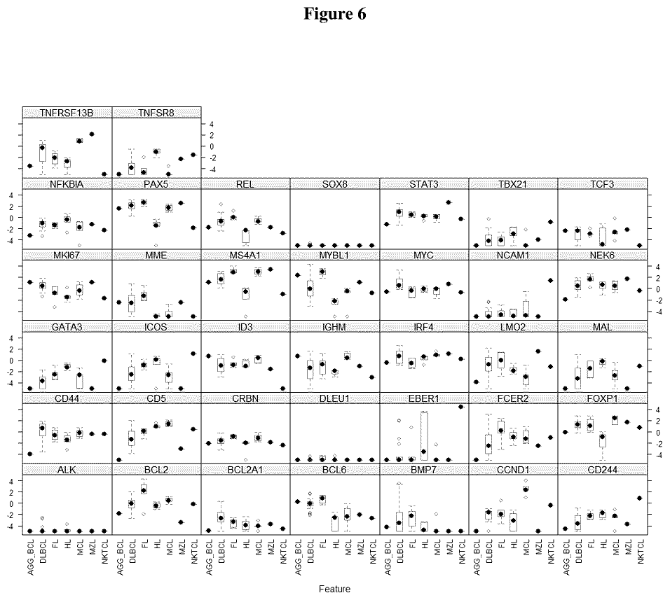

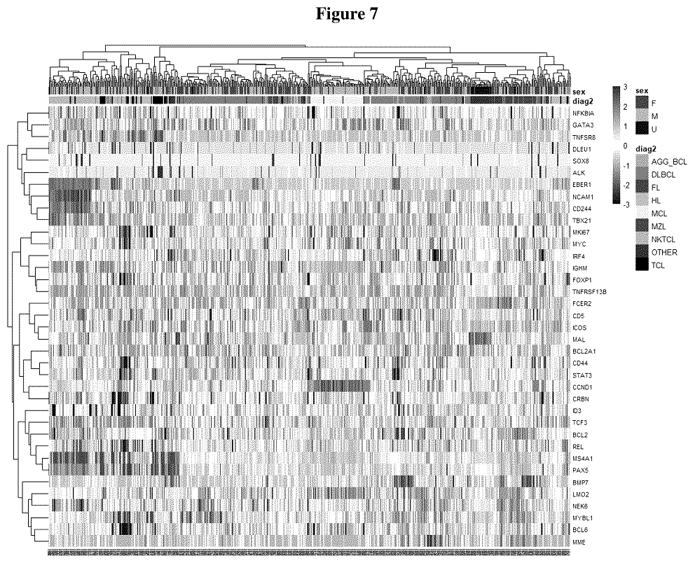

FIG. 4 is a plot of the Spearman correlation coefficients for each gene in the panel of genes. FIGS. 5 and 6 are boxplots (median plotted with 25th and 75th percentiles shown as the bottom and top of the boxes) for the diagnostic and relapse samples by lymphoma subtype. FIG. 7 is a heatmap with hierarchical clustering using Spearman correlation as the distance function with complete linkage of the diagnostic samples and for the diagnostic DLBCL sample. FIG. 7 demonstrates how different lymphoma subtypes may cluster together based on the genes selected, suggesting these genes are good candidates for differentiating lymphoma subtypes.

The class label with the highest probability value was assigned. Calls based on averaged probabilities were categorized as indeterminate (probability <0.60) and diagnostic (probability >0.60). The high probability overall assay accuracy was 94%. See FIG. 9.

### Example 2—Prophetic Example—Dual Clinical Scenario Use for Breast Cancer

After adding all desired genes associated with lymphoma sub types additional space on the assay was leveraged to add genes that would allow the same method to determine the presence or absence of three breast cancer genes ESR1 (estrogen receptor), PGR (progesterone receptor) and ERBB2 (HER2/Neu receptor).

Physician reported suspected diagnoses of breast cancer from a statistically significant number of patients will be identified and reviewed. Samples from the patients will be evaluated to determine, by World Health Organization pathology standards, the true diagnosis of breast cancer subtype.

These potential diagnoses will then be matched to gene expression data generated by a panel of genes most likely to differentiate the different potential diagnoses. Formalin-fixed, paraffin-embedded samples will be collected from the patients and subjected to multiplex PCR and standard capillary electrophoresis to determine the presence or absence of one or more genes from the panel of genes. The collected data will be input into multiple machine learning approaches to generate a diagnostic algorithm.

This diagnostic approach will be validated on an independent cohort of samples. Ultimately, the diagnostic algorithm will be able to distinguish between breast cancer subtypes ER/PR⁺HER2, ER/PR⁺HER2⁺, ER/PR⁻HER2⁺, or ER/PR⁻HER2⁻.

Various aspects of the present disclosure may be used alone, in combination, or in a variety of arrangements not specifically discussed in the embodiments described in the foregoing and is, therefore, not limited in its application to the details and arrangement of components set forth in the foregoing description or illustrated in the drawings. For example, aspects described in one embodiment may be combined in any manner with aspects described in other embodiments.

1. A method of diagnosing lymphoma, breast cancer, a lymphoma subtype or a breast cancer subtype, in a patient comprising:
(a) detecting the presence or absence of one or more genes from a panel of genes associated with lymphoma, breast cancer, a lymphoma subtype, and/or a breast cancer subtype in a sample from a patient; and
(b) categorizing the sample into one or more diagnostic categories thereby diagnosing the lymphoma and/or lymphoma subtype, or breast cancer and/or breast cancer subtype;
wherein the panel of genes comprises one or more of BACH2, BCMA, CD22, CD38, CD43, CD79a, CD138, CD200, CD274/PD-L1, CK7, CK20, Cyp141, ESR1, HER2/neu, HHV8, HBZ(HTLV), IS6110, PGR, TTF-1, LANA-1, and TDT or any combination thereof and wherein each gene of the panel of genes is associated with one or more diagnostic categories.

2. The method of claim 1, wherein the sample comprises a tissue volume of about 0.25 mm³ to about 5 mm³.

3. The method of claim 1, wherein the sample is a formalin-fixed, paraffin-embedded (FFPE) sample.

4. The method of claim 1, wherein the detecting is performed using multiplex PCR and standard capillary electrophoresis.

5. The method of claim 1, wherein the panel of genes further comprises one or more of ALK, BCL2, BCL2A1, BCL6, BMP7, CCND1, CD244, CD44, CD5, CRBIN, DLEU1, EBER1, FCER2, FOXP1 GATA3, ICOS, ID3, IGHM, IRF4, LMO2, MAL, MKI67, MME, MS4A1, MYBL1, MYC, NCAM1, NEK6, NFKBIA, PAX5, REL, SOX8, STAT3, TBX21, TCF3, TNFRSF13B, and TNFSR8 or any combination thereof.

6. The method of claim 1, wherein the diagnostic categories comprise one or more of anaplastic large cell lymphoma, ALK− or ALK+, angioimmunoblastic T-cell lymphoma, B-lymphoblastic lymphoma, Burkitt lymphoma EBV− or EBV+, chronic lymphocytic leukemia/SLL, classical Hodgkin lymphoma, diffuse large B-cell (DLBCL), EBV+DLBCL, extranodal NK/T cell lymphoma, grade I-IIIA follicular lymphoma, grade IIIB follicular lymphoma, HHV− or HHV+Castleman Disease, Kaposi sarcoma, Mantle Cell lymphoma (MCL), marginal zone lymphoma (MZL), Mycosis Fungoides, nodal hyperplasia, nodular lymphocytes-predominant Hodgkin lymphoma, peripheral T-cell lymphoma NOS, plasma cell neoplasm, Plasmablastic lymphoma, T-cell lymphoblastic lymphoma, or tuberculosis.

7. The method of claim 6, wherein the method is one for diagnosing breast cancer subtypes and the diagnostic categories further comprise ER/PR+HER2, ER/PR+HER2+, ER/PR−HER2+, or ER/PR−HER2−.

8. The method of claim 1, further comprising generating a probability threshold.

9. The method of claim 8, further comprising calculating a diagnostic accuracy.

10. The method of claim 9, wherein, when the probability threshold is at least 0.6, the diagnostic accuracy is at least about 94%.

11. The method of claim 1, further comprising administrating a therapeutically effective amount of treatment to the patient, wherein the treatment is selected from a therapeutic agent, radiation therapy, or surgery.

12. The method of claim 11, wherein the therapeutic agent comprises one or more of a pharmaceutical agent, chemotherapeutic agent, an immunotherapeutic agent or combinations thereof.

13. A kit comprising:
(a) one or more reagents for detecting the presence or absence of one or more genes from a panel of genes associated with lymphoma, breast cancer, a lymphoma subtype, and/or a breast cancer subtype in a sample from a patient;
(b) instructions for detecting the presence or absence of the one or more genes; and
(c) a classification guide for categorizing the sample into one or more diagnostic categories; wherein the panel of genes comprises one or more of BACH2, BCMA, CD22, CD38, CD43, CD79a, CD138, CD200, CD274/PD-L1, CK7, CK20, Cyp141, ESR1, HER2/neu, HHV8, HBZ(HTLV), IS6110, PGR, TTF-1, LANA-1, and TDT or any combination thereof and wherein each gene of the panel of genes is associated with one or more of the diagnostic categories.

14. The kit of claim 13, wherein the diagnostic categories comprise anaplastic large cell lymphoma, ALK− or ALK+, angioimmunoblastic T-cell lymphoma, B-lymphoblastic lymphoma, Burkitt lymphoma EBV− or EBV+, chronic lymphocytic leukemia/SLL, classical Hodgkin lymphoma, diffuse large B-cell (DLBCL), EBV+DLBCL, extranodal NK/T cell lymphoma, grade I-IIIA follicular lymphoma, grade IIIB follicular lymphoma, HHV− or HHV+Castleman Disease, Kaposi sarcoma, Mantle Cell lymphoma (MCL), marginal zone lymphoma (MZL), Mycosis Fungoides, nodal hyperplasia, nodular lymphocytes-predominant Hodgkin lymphoma, peripheral T-cell lymphoma NOS, plasma cell neoplasm, Plasmablastic lymphoma, T-cell lymphoblastic lymphoma, or tuberculosis.

15. The kit of claim 14, wherein the diagnostic categories further comprise breast cancer subtypes ER/PR⁺HER2⁻, ER/PR⁺HER2⁺, ER/PR⁻HER2⁺, or ER/PR⁻HER2⁻.
